(19) United States
(12) Patent Application Publication
(10) Pub. No.: US 2013/0210028 A1
(43) Pub. Date: Aug. 15, 2013

(54) Biological Assays Using Microparticles

(71) Applicant: Affymetrix, Inc., (US)

(72) Inventors: Jared C. Pache, Tracy, CA (US); Lawrence Greenfield, Eugene, OR (US)

(73) Assignee: Affymetrix, Inc., Santa Clara, CA (US)

(21) Appl. No.: 13/720,359

(22) Filed: Dec. 19, 2012

Related U.S. Application Data
(60) Provisional application No. 61/578,405, filed on Dec. 21, 2011.

(51) Int. Cl. G01N 33/566

(52) U.S. Cl. CPC G01N 33/566
USPC 435/7.2

(57) ABSTRACT

Encoded microparticles described are useful in the study of many different biological agents in multiplex assays. For instance, the encoded microparticles may be employed in various co-precipitation assays to purify and/or isolate various analytes of interest. Encoded microparticles may also be used as real-time detectors in many different situations whereby binding of a secreted analyte or contaminating analyte may be detected using various labeling techniques. Further, encoded microparticles may be attached in a specific manner to particular cell types, for instance in a heterogeneous mixture of cells, either fixed in tissue or circulating, to allow identification, localization and/or sorting of the cells in the context of various biological events under various environments or conditions.

### RELATED APPLICATIONS

The present application claims priority from U.S. Provisional Patent Application Ser. No. 60/578,405, filed Dec. 21, 2012 and is hereby incorporated by reference herein in its entirety for all purposes.

### FIELD

The present invention relates to the art of microparticles, and more particularly to encoded microparticles and methods of use thereof in biological assays.

### BACKGROUND

Microparticles or nanoparticles are often referred to as structures whose characteristic dimensions are on the order of micrometers or less, such as those with volumes of 1 mm³ or less. Due to their unique properties arising from their small characteristic dimensions, microparticles have found distinguishable applications in laboratory research and many industrial fields. Encoded microparticles possess a means of identification and are an important subclass of the general field of microparticles. Because encoded particles carry information and can be physically tracked in space and time, they greatly extend the capabilities of non-encoded particles. One important application for encoded microparticles is multiplexed bioassays, including those involving DNA, proteins and cells. These assays can be diagnostic or predictive. The encoded aspect of encoded microparticles provides great flexibility in detecting multiple targets in a single assay.

Encoded microparticles and their manufacture have previously been reported. See, for instance, U.S. Pat. Nos. 7,745,091, 7,745,092, and U.S. Patent Application Publication Nos. US20100290018, US20100227279, US20100227770, US20100297336, 20100075438 and US20100297448, and U.S. patent application Ser. Nos. 11/502,606 and 11/580,514 (all of which are incorporated herein by reference in their entirety for all purposes).

For many applications, encoded microparticles offer the advantages of a large number of identifiable codes (i.e. a high codespace), accurate and reliable identification of the encoded particles, material compatibility for a particular application, low cost manufacturing of the microparticles (on a per batch, per particle, and per code set basis), and flexibility in the detection systems. In this sense, the code in the encoded microparticle may obviate the requirement of many different colors or types of labels for each target, thus remarkably simplifying any multiplex biological assay. Obtaining multiple fluorescent labels which work optimally together in a single assay can be very challenging. (See, Baker, Monya, “Bright Light, Better Labels,” Nature, 478:137-142, 2011).

On the other hand, many different types of labels exist and may be utilized in biological assays. Combining various labeling strategies with encoded microparticles provides additional flexibility and opportunities to detect and measure otherwise intractable analytes. Addition of labels to encoded microparticles in multiplex biological assays adds an additional layer of functionality and flexibility to design of such assays. There are various protein/enzyme-based labels chemical labels. For instance, the molecular beacon technology is a robust and unique technique able to detect interaction between a bait molecule and target molecule (Sigma-Aldrich, see, Tyagi et al., Nature Biotechnology, 14(3):303-308, 1996 and Tyagi et al., Nature Biotechnology, 18(11):1191-1196, 2000). Various other labels include chemiluminescent and phosphorescent labels, quantum dots, light-scattering labels (e.g., colloidal gold particles), or an enzyme (e.g., HRP) and any fluorescence resonance energy transfer (FRET) pairs as in the commercially available cameleon calcium sensors (Invitrogen/Life Technologies, see BioProbes, 55:26-28, 2008, and Palmer et al., Nat. Protocol., 1(3):1057-1065, 2006) which utilizes the FRET arising from interaction/proximity between cyan-fluorescent protein (CFP) and yellow-fluorescent protein (YFP). Many other FRET pairs have been reported. (See, Pollok et al., 1999, “Using GFP in FRET-based applications,” Trends Cell Biol. 9(2):57-60 and Selvin P R, 2000, “The renaissance of fluorescence resonance energy transfer,” Nat. Struct. Biol., 7(9):730-4). Various other dye-based labels are known in the art, such as Alexa Fluor Dyes (Life Technologies, Inc., California, USA, available in a wide variety of wavelengths, see for instance, Panchuk, et al., J. Hist. Cyto., 47:1179-1188, 1999), biotin-based dyes, digoxigenin, AttoPhos (JBL Scientific, Inc., California, USA, available in a variety of wavelengths, see for instance, Cano et al., Biotechniques, 12(2):264-269, 1992), ATTO dyes (Sigma-Aldrich, St. Louis, Mo.), or any other suitable label. Other newer techniques for measuring interaction include such methods as back-scattering interferometry (BSI, see Bornhop et al., Screening, 10:14-16, 2009).

Further, label amplification technologies exist which may be coupled with encoded microparticle use to provide additional functionality to the assays. For instance, the QuantiGene® technology (Affymetrix, Inc.) provides a simple and efficient means of significantly amplifying a single analyte capture event. Based on a branched-DNA methodology, this amplification technique has been highlighted in, for instance, U.S. Pat. Nos. 7,803,541, 7,927,798, 7,968,327, 7,615,351, and 7,951,539, and related US patents and patent applications (incorporated herein by reference in their entirety for all purposes).

Analytes of particular interest for diagnostics and other applications include, for instance microRNA and miRNA molecules, circulating tumor cells, secreted cellular components and biochemical signals, specific cell types and determination and quantitation thereof, and various protein-protein interactions indicative of disease states, etc. All of these biological interactions and events may be monitored more accurately, and in some cases in real time, for the first time utilizing the compositions and methodologies disclosed herein.

Therefore, what is desired is an encoded microparticle or a set of encoded microparticles carrying coded information and compositions, kits and methods of their utilization in biological assays designed to detect and measure biological events, interactions and analytes of interest.

### SUMMARY

Encoded microparticles and methods for using them in biological assays are provided. In one non-limiting example, a method of detecting an analyte, is disclosed, wherein a set of encoded microparticles are provided which comprises two or more subsets of encoded microparticles, each of the two or more subsets of encoded microparticle comprising a spatial code unique to each subset. Attached to each subset of the two or more subsets of encoded microparticles is a unique molecule possessing a specificity for a unique binding partner molecule. The unique molecules may optionally be labeled. The encoded microparticles are then incubated with a sample suspected of comprising an analyte. Thus, the label may be detected and the code associated with the detected label determined. The presence of the analyte may be determined based on the unique molecule corresponding to the determined code of the encoded microparticle.

Further non-limiting examples include methods of identifying the presence of a cell, or group of cells, based on inherent characteristics of each cell. Inherent characteristics of the cell may include such elements as the presence or absence of expressed extracellular markers, such as receptors and other cell membrane embedded molecules. The method begins as described above in the previous assay, and the sample comprising or suspected of comprising the cell(s) is incubated with the encoded microparticles. If the cell is found to be labeled, the code on the encoded microparticle may be determined and then the cell type deduced based on the code and the corresponding molecule attached to the microparticle. If no cell is present, none of the labeled microparticles will bind to the sample and be detected.

### BRIEF DESCRIPTION OF DRAWINGS

While the appended claims set forth the features of the present invention with particularity, the invention, together with its objects and advantages, may be best understood from the following detailed description taken in conjunction with the accompanying drawings of which:

FIG. 1A schematically illustrates an encoded microparticle of the invention;

FIG. 1b is a side view cross-section of the microparticle in FIG. 1a;

FIG. 2 schematically illustrates another example encoded microparticle of the invention.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a molecule” includes a plurality of such molecules, and the like.

The term “about” as used herein indicates the value of a given quantity varies by +/−10% of the value, or optionally +/−5% of the value, or in some embodiments, by +/−1% of the value so described.

The term “polynucleotide” (and the equivalent term “nucleic acid”) encompasses any physical string of monomer units that can be corresponded to a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA, such as 2′-O-methylated oligonucleotides), and the like. The nucleotides of the polynucleotide can be deoxyribonucleotides, ribonucleotides or nucleotide analogs, can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The polynucleotide can additionally comprise non-nucleotide elements such as labels, quenchers, blocking groups, or the like. The polynucleotide can be, e.g., single-stranded or double-stranded.

The term “analog” in the context of nucleic acid analog is meant to denote any of a number of known nucleic acid analogs such as, but not limited to, LNA, PNA, etc. For instance, it has been reported that LNA, when incorporated into oligonucleotides, exhibit an increase in the duplex melting temperature of 2° C. to 8° C. per analog incorporated into a single strand of the duplex. (See, for example, Singh et al., Chem. Commun., 1998, 4:455-456; Koshkin et al., Tetrahedron, 1998, 54:3607-3630; Wahlestedt et al., Proc. Natl. Acad. Sci. U.S.A., 2000, 97:5633-5638; Kumar et al., Bioorg. Med. Chem. Lett., 1998, 8:2219-2222; Wengel et al., PCT International Application Number PCT/DK98/00303 which published as WO 99/14226 on Mar. 25, 1999; Singh et al., J. Org. Chem., 1998, 63:10035-10039, the text of each is incorporated by reference herein, in their entirety). Examples of issued US Patents and Published U.S. Patent Applications disclosing various bicyclic nucleic acids include, for example, U.S. Pat. Nos. 6,770,748, 6,268,490 and 6,794,499 and U.S. Patent Application Publication Nos. 20040219565, 20040014959, 20030207841, 20040192918, 20030224377, 20040143114, 20030087230 and 20030082807, the text of each of which is incorporated by reference herein, in their entirety. (See also, for example: Mikhailov et al., Nucleosides and Nucleotides, 1991, 10:393-343; Saha et al., J. Org. Chem., 1995, 60:788-789; Beigleman et al., Nucleosides and Nucleotides, 1995, 14:901-905; Wang, et al., Bioorganic & Medicinal Chemistry Letters, 1999, 9:885-890; and PCT Internation Application Number WO94/22890 which was published Oct. 13, 1994, and Tarkoy et al., Helv. Chim. Acta, 1993, 76:481; Tarkoy and C. Leumann, Angew. Chem. Int. Ed. Engl., 1993, 32:1432; Egli et al., J. Am. Chem. Soc., 1993, 115:5855; Tarkoy et al., Helv. Chim. Acta, 1994, 77:716; M. Bolli and C. Leumann, Angew. Chem., Int. Ed. Engl., 1995, 34:694; Bolli et al., Helv. Chim. Acta, 1995, 78:2077; Litten et al., Bioorg. Med. Chem. Lett., 1995, 5:1231; J. C. Litten and C. Leumann, Helv. Chim. Acta, 1996, 79:1129; Bolli et al., Chem. Biol., 1996, 3:197; Bolli et al., Nucleic Acids Res., 1996, 24:4660, Jones et al., J. Am. Chem. Soc., 1993, 115:9816 and, U.S. Pat. Nos. 7,572,582, 7,399,845, 7,034,133, 6,794,499 and 6,670,461, all of which are incorporated herein by reference in their entirety for all purposes).

A “polynucleotide sequence” or “nucleotide sequence” is a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined. Alternative terms include, for instance, oligonucleotides. Such terms are meant to encompass all forms of nucleic acid polymers, including, but not limited to, mRNA, microRNA, miRNA, siRNA, mitochondrial RNA, DNA, gDNA, DNA analogs, and the like.

Two polynucleotides “hybridize” when they associate to form a stable duplex, e.g., under relevant assay conditions. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, N.Y.).

The term “complementary” refers to a polynucleotide that forms a stable duplex with its “complement,” e.g., under relevant assay conditions. Typically, two polynucleotide sequences that are complementary to each other have mismatches at less than about 20% of the bases, at less than about 10% of the bases, preferably at less than about 5% of the bases, and more preferably have no mismatches.

A “label” is a moiety that facilitates detection of a molecule. Common labels in the context of the present invention include fluorescent, luminescent, light-scattering, and/or colorimetric labels. Suitable labels include radionuclides, dyes, FRET pairs, substrates, cofactors, inhibitors, chemiluminescent moieties, quantum dots, phosphorescent species, and the like. Patents teaching the use of such labels include U.S. Pat. Nos. 3,817,837; 3,850,752; 3,939,350; 3,996,345; 4,277,437; 4,275,149; and 4,366,241. Several additional labels are commercially available and can be used in the context of the invention. There are various protein/enzyme-based labels chemical labels. For instance, the molecular beacon technology is a robust and unique technique able to detect interaction between a bait molecule and target molecule (Sigma-Aldrich, see, Tyagi et al., Nature Biotechnology, 14(3):303-308, 1996 and Tyagi et al., Nature Biotechnology, 18(11):1191-1196, 2000). Various other labels include chemiluminescent and phosphorescent labels, quantum dots, light-scattering labels (e.g., colloidal gold particles), or an enzyme (e.g., HRP) and any fluorescence resonance energy transfer (FRET) pairs as in the commercially available cameleon calcium sensors (Invitrogen/Life Technologies, see BioProbes, 55:26-28, 2008, and Palmer et al., Nat. Protocol., 1(3):1057-1065, 2006) which utilizes the FRET arising from interaction/proximity between cyan-fluorescent protein (CFP) and yellow-fluorescent protein (YFP). Many other FRET pairs have been reported. Various other dye-based labels are known in the art, such as Alexa Fluor Dyes (Life Technologies, Inc., California, USA, available in a wide variety of wavelengths, see for instance, Panchuk, et al., J. Hist. Cyto., 47:1179-1188, 1999), biotin-based dyes, digoxigenin, AttoPhos (JBL Scientific, Inc., California, USA, available in a variety of wavelengths, see for instance, Cano et al., Biotechniques, 12(2):264-269, 1992), ATTO dyes (Sigma-Aldrich, St. Louis, Mo.), or any other suitable label. Other newer techniques for measuring interaction include such methods as back-scattering interferometry (BSI, see Bornhop et al., Screening, 10:14-16, 2009).

“Microparticles” include particles having a code, including sets of encoded microparticles. (See, for instance, U.S. patent application Ser. No. 11/521,057, allowed, which is incorporated herein by reference in its entirety for all purposes). Such encoded microparticles may have a longest dimension of 50 microns, an outer surface substantially of glass and a spatial code that can be read with optical magnification. A microparticle may be cuboid in shape and elongated along the Y direction in the Cartesian coordinate. The cross-sections perpendicular to the length of the microparticle may have substantially the same topological shape—such as square shape. Microparticles may have a set of segments and gaps intervening the segments in parallel along the axis of the longest dimension if the microparticle is rectangular. Specifically, segments with different lengths (the dimension along the length of the microparticle, e.g. along the Y direction) may represent different coding elements; whereas gaps preferably have the same length for differentiating the segments during detection of the microparticles. The segments of the microparticle may be fully enclosed within the microparticle, i.e. completely encapsulated by a surrounding outer layer which may be silicon/glass. As an alternative feature, the segments can be arranged such that the geometric centers of the segments are aligned to the geometric central axis of the elongated microparticle. A particular sequence of segments and gaps thereby represent a code within each micoparticle. The codes may be derived from a pre-determined coding scheme thereby allowing identification of the microparticle. The microparticles may additionally have various structural aberrations, such as tags or tabs, on one or more ends, thus allowing for a two-fold or more increase in code space. The microparticles may also be present as a “bi-particle” wherein the microparticle actually comprises two or more particles stuck together, i.e. missing the last etching step so as to allow two particles to remain attached together with an intervening material between them comprised of material consistent with the coating present on the rest of the microparticle. (See, for instance, U.S. Pat. Nos. 7,745,091, 7,745,092, and U.S. Patent Application Publication Nos. US20100290018, US20100227279, US20100227770, US20100297336, 20100075438 and US20100297448, and U.S. patent application Ser. Nos. 11/502,606, 11/580,514, and 12/779,413, incorporated herein by reference in its entirety for all purposes).

A “microorganism” is an organism of microscopic or submicroscopic size. Examples include, but are not limited to, bacteria, fungi, yeast, protozoans, microscopic algae (e.g., unicellular algae), viruses (which are typically included in this category although they are incapable of growth and reproduction outside of host cells), subviral agents, viroids, and mycoplasma.

A variety of additional terms are defined or otherwise characterized herein.

### DETAILED DESCRIPTION

### Encoded Microparticles

An encoded microparticle is provided carrying a code, and a set of encoded microparticles are provided with distinguishable codes, wherein the codes comply with a pre-determined coding scheme. Preferably, the microparticles in the examples below have a volume of 1 mm³ or less. The microparticle of the invention enables fast, precise and less complicated detection of the code. The encoded microparticles are those which have been described above in, for instance, U.S. Pat. Nos. 7,745,091, 7,745,092, and U.S. Patent Application Publication Nos. US20100290018, US20100227279, US20100227770, US20100297336, 20100075438 and US20100297448, and U.S. patent application Ser. Nos. 11/502,606, 11/580,514, and 12/779,413, incorporated herein by reference in its entirety for all purposes.

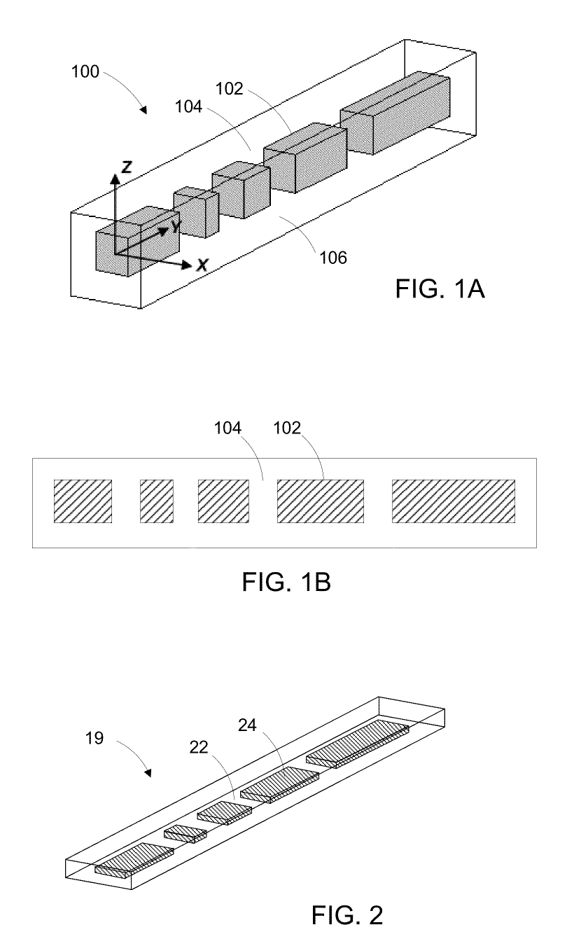

As an example, FIG. 1A schematically illustrates an encoded microparticle of the invention. Microparticle 100 is a cuboid structure elongated along the Y direction in the Cartesian coordinate as shown in the figure. The cross-sections perpendicular to the length of the microparticle have substantially the same topological shape—which is square in this example. The microparticle in this particular example has a set of segments (e.g. segment 102) and gaps (e.g. gap 104) intervening the segments. Specifically, segments with different lengths (the dimension along the length of the microparticle, e.g. along the Y direction) represent different coding elements; whereas gaps preferably have the same length for differentiating the segments during detection of the microparticles. The segments of the microparticle in this example are fully enclosed within the microparticle, for example within body 106. As an alternative feature, the segments can be arranged such that the geometric centers of the segments are aligned to the geometric central axis of the elongated microparticle. A particular sequence of segments and gaps represents a code. The codes are derived from a pre-determined coding scheme.

Segments of the microparticle can be any suitable form. In an example of the invention, each segment of the microparticle has a substantially square cross-section (i.e. the cross-section in the X-Z plane of a Cartesian coordinate as shown in FIG. 1A) taken perpendicular to the length (i.e. along the Y direction in the Cartesian coordinate in FIG. 1A) of the microparticle. The segments may or may not be fabricated to have substantially square cross-section. Other shapes, such as rectangular, circular, and elliptical, jagged, curved or other shapes are also applicable. In particular, the code elements—i.e. segments and gaps, may also take any other suitable desired shape. For example, the segment (and/or the gaps) each may have a cross-section that is rectangular (e.g. with the aspect ratio of the rectangular being 2:1 or higher, such as 4:1 or higher, 10:1 or higher, 20:1 or higher, or even 100:1 or higher, but preferably less than 500:1).

The microparticle example of FIG. 1A has six major surfaces, namely surfaces of (X=±x₀, Y, Z), surfaces (X, Y, Z=±z₀), and surfaces (X, Y=±y₀, Z), wherein x₀, y₀, and z₀ are respectively the width, length, and height of the microparticle. According to the invention, at least two of the above six surfaces X=±x₀ (or surfaces Z=±z₀), more preferably four of the above six major surfaces X=±x₀, surfaces Z=±z₀ are substantially continuous, regardless of whether each surface has or does not have indentations. With this configuration, the microparticle exhibits substantially the same geometric appearance and specific properties to the detector—such as an optical imaging apparatus. In fact, the major surfaces can be made substantially flat. For example, even though roughness or varying profiles may be caused during fabrication, substantially flat major surfaces can still be obtained using standard surface machining techniques, such as over-deposit and etch back or chemical-mechanical-polishing (CMP) techniques, as well as proper control of patterning steps to create smooth vertical sidewall profiles.

Though the microparticle may have the same length in the X, Y and/or Z directions, preferably the encoded microparticle has a ratio of the length to width of from 2:1 to 50:1, e.g. from 4:1 to 20:1. In an example of the invention, the microparticle has a length (e.g. the dimension along the Y direction) of 70 microns or less, 50 microns or less, 30 microns or less, such as 20 microns or less, 16 microns or less, or even 10 microns or less. The width (e.g. the dimension along the X direction), as well as the height (the dimension along the Z direction), of the microparticle can be 15 microns or less, 10 microns or less, 8 microns or less, 4 microns or less, or even 1 microns or less, such as 0.13 micron. Widths as small as from 0.5 to 2 microns are also possible. Other than the shape as shown in FIG. 1A and discussed above, the microparticle may take a form of rod, bar, disk or any other desired shapes.

For representing a code derived from the predetermined coding scheme, the segments and gaps are arranged along the length (the Y direction) of the elongated microparticle (2D, or even 3D, arrangements however are also possible). Specifically, the segments and gaps are alternately aligned along the length with the each segment being separated (possibly fully separated and isolated) by adjacent gaps; and each gap is separated (possibly fully separated and isolated) by adjacent segments, which is better illustrated in a cross-sectional view in FIG. 1B, which will be discussed in the following.

In an example of the invention, any suitable number of segments can be used—e.g. from 2 to 20, or more typically from 3 to 15 segments (more typically from 3 to 8 segments) of less transparent material (as compared to the intervening gaps between the segments) are provided within the encoded microparticle. To form the code, it is possible that the segments of less transparent material are varying lengths. Alternatively, the segments of less transparent material could each have substantially the same length whereas the intermediate segments of more transparent material could have varying lengths. Of course, the segments of more transparent material and the intermediate segments of less transparent material could both have varying lengths in order to represent the code.

Referring to FIG. 1B, the cross-section is taken in the Y-Z plane (or equivalently in the X-Y plane) of the particle in FIG. 1A. Segments (e.g. segment 102) and gaps (e.g. gap 104) alternate along the length of the microparticle.

The microparticle may have a six sided shape with four elongated sides and two end sides. The encoded microparticle can be configured such that the code of the encoded microparticle can be detectable regardless of which of the four elongated sides the barcode is disposed on. The microparticle may have a ratio of the length to width is from 2:1 to 50:1, from 4:1 to 20:1. The length of the microparticle is preferably from 5 to 100 μm and more preferably less than 50 um. The width of the microparticle can be from 0.5 to 10 μm. In other examples, the length of the microparticle can be less than 10 μm, less than 25 μm, less than 25 μm; less than 5 μm, less than 27 μm; and the width of the microparticle can be less than 3 um. The ratio of width to height of the microparticle can be from 0.5 to 2.0. The ratio of the length to width of the microparticle can be from 2:1 to 50:1. The cross section taken along the length of the microparticle is substantially rectangular with a length at least twice the width.

The encoded microparticles may be, for instance, 2.4×2.4×20.8 microns, or 5.6×1.8×20.8 microns, or 1.6×1.6×16 microns in dimensions. Alternatively, the encoded microparticles may be 1.0, 1.1, 1.2, 1.3, 1.4, 1.5, 1.6, 1.8, 1.9, 2.0, 2.1, 2.2, 2.3, 2.4, 2.5, 2.6, 2.7, 2.8, 2.9, or as many as 3 microns in the X or Z dimension in FIG. 1A, and as many as 10, 11, 12, 13, 14, 15, 16, 17, 18, 19, 20, 21, 22, 23, 24, 25, 26, 27, 28, 29 or even 30 microns in the Y direction of FIG. 1A, or any combination thereof. The encoded microparticles may be single bar-type microparticles as depicted in FIGS. 1 and 2, or “twin” particles in which two such particles are stuck together with two identical codes or two different codes to a single particle having twice the dimension lengths of a single particle.

The microparticle may have a glass body with segments embedded therein. The difference of the transmissivity of the glass body and segments can be 10% or more. The glass body may have a length of less than 50 um and a width of less than 10 um with the glass body having a volume of from 5 to 500 μm³. The encoded microparticle may have 2 to 15, 3 to 10, or 4 to 8 portions of less transparent material within the encoded microparticle. The code incorporated in the microparticle can be binary or non-binary or any other desired codes. The microparticle may have biochemical molecules attached to one or more surfaces of the microparticle, such as DNA and RNA probes with a density of from 10² to 10⁶/μm². When fabricated on the wafer-level, the wafer may have a surface area of from 12.5 in² to 120 in², and wherein there are at least 3 million microparticles per in² of the wafer. The wafer may have at least one million codes are formed on the substrate, or at least two hundred different codes are present within the one million codes, or at least 3000 different codes are present within the one million codes. When placed in a liquid buffer, for example in a bioassay, the microparticles can form a single monolayer with a 2 dimensional diffusion coefficient of the microparticles greater than 1×10⁻¹² cm²/s and more preferably greater than 1×10⁻¹¹ cm²/s.

### Biological Assays

In biological applications, the microparticles are often used to carry biochemical probe molecules. For immobilizing such probe molecules, the microstructure preferably comprises a surface layer, such as a silicon dioxide layer, which can be chemically modified to attach to the probe molecules. In accordance with an example of the invention, the microparticles are constructed such that the microparticles are capable of forming a monolayer, for example, at the bottom of a well containing a liquid; and the monolayer comprises 500 or more particles per square millimeter, more preferably 1,000 or more, 2,000 or more, or 3,000 or more microparticles per square millimeter. In an alternative example, the microparticles can form a monolayer that such that the detectable particles occupy 30% or more, 50% or more, or 70% or more of the total image area, i.e. the image field of view. In connection with the example mechanism of self-assembled monolayer formation, it is preferred that the 2D diffusion coefficient of the microparticles of the invention is greater than 1×10⁻¹² cm² per second. For accommodating the monolayer of the microparticles, the container for holding the microparticles in detection preferably has a substantially flat bottom portion.

Imaging the microparticles may be accomplished in any number of ways including, for instance, a CCD camera, an inverted epi-fluorescence microscope configuration, or a confocal microscope system, Total Internal Reflection Fluorescent (TIRF), etc. as previously described in the references cited above, incorporated herein by reference. The encoded microparticles may be detected using both brightfield and reflected light. The encoded microparticles may be imaged in a static field, or in a flow-based apparatus, similar to those described in, for instance, U.S. Patent Application Publication Nos. 20100075438. Other flow-type instrumentation including a flow cell such as commonly used in flow cytometry applications may also be used to read the code of the microparticles as they fly through the flow cell. (See, for instance, Nolan, et al., Trends in Biotechnology, 20(1):9-12, 2002, and Wilson et al., “Encoded Microcarriers for High-Throughput Multiplexed Detection,” Angew. Chem. Int. Ed., 45(37):6104-6117, 2006, especially at pages 6110-6111 and FIGS. 6 and 7, both of which are incorporated herein by reference in their entireties for all purposes).

A flow-cell enabling the microparticles flowing in a fluid can be provided for detection by continuous imaging. Transmission images, or forward scatter (fluorescence measured from the side at a longer wavelength), and fluorescence image pairs may be acquired with an optical system using a flow cell. Encoded microparticles can flow in a carrier fluid. Flow may be driven by pressure (hydrodynamic) or electrical means (electro-phoretic or electro-osmotic). Further, microparticles may be aligned with electric or magnetic fields. By appropriately matching the flow speed, flow cell size, and optical system, all particles passing through the flow cell can be imaged, thereby providing a system for high throughput detection working in concert with imaging software which can capture images of the particles and resolve their codes. Another exemplary system for high throughput flow based detection of the encoded microparticles of the invention is a flow cytometer, the methods and applications thereof are well known in the art. If the encoded microparticles are magnetic, optionally the encoded microparticles may be focused into a flow stream before entering the flow chamber using electromagnetic principles, etc.

The encoded microparticles, systems, and methods of the invention have a wide range of applications in the fields of biology, chemistry, and medicine, as well as in security and commercial fields involving the tagging of monetary bills, identification cards and passports, commercial products, and the like. In one example, the microparticles can be used in for molecular detection, such for as analyzing DNA, RNA, and proteins. In other examples, combinatorial chemistry or drug screening assays are performed as known in the art.

Biomolecules, such as DNA or RNA, may be immobilized on the surface of the encoded microparticles and referred to as “probes”. Each species of probe is immobilized onto a differently encoded microparticle and a lookup table may be generated for future reference to determine which code corresponds with which probe. Each species of probe also has one or more corresponding species of “targets” for which the binding between the two is specific. The probe/target terminology is usually used in reference to DNA and RNA complements but in this context refers to all biomolecules, including antibodies, proteins, cells, etc. Many probes are immobilized on a single particle, typically with a density on the order 10⁴/μm² or higher. The singular use of “a probe” often refers to a plurality of probe molecules; and “a code” often refers to a plurality of particles of a certain code, as with other terms used herein.

The mating of the encoded particles and biomolecules produces a “pooled probe set”. The pooled probe set is a mixture of encoded particles where each code has a particular probe attached to the particle surface. The pooled probe set can then be used to determine the amount of individual targets present in a mixture of targets. The mixture of targets is referred to as the sample and is typically derived from a biological specimen. The sample, in some embodiments, may be labeled. When the sample is mixed with the pooled probe set, the probes and targets find each other in solution and bind together. After the reaction, the particles are imaged to read the codes and quantify the optional label signal.

The samples reacted with the microparticles may be a purified biological extract or a non-purified sample, including but not limited to whole blood, serum, cell lysates, swabs, FFPE tissue slices, live tissue cultures or tissue extracts. Samples may be from any biological source, including, but not limited to animals, viruses, microorganisms. Samples may be, for example, from a human source. Samples may include purified or partially-purified DNA, RNA, and protein and mixtures thereof. Samples may also be total RNA isolated from, for instance, a cell line, said cell lines being transformed, cancerous or immortalized, or alternatively infected with a microorganism or virus, or originating from a tumor, for example. The samples reacted with the microparticles may be produced by culturing, cloning, dissection, or microdissection. Cells may serve as either the sample or probe in a bioassay utilizing the microparticles and other aforementioned inventions.

It is noted that multiple different samples may be identified in a single bioassay as discussed above. Before the detection and after the hybridization, as in the case of oligonucleotides analytes, the microparticles can be placed into wells of a well plate or other container for detection. In one detection example, the microparticles settle by gravity onto the bottom surface of the well plate. The microparticles in the well can be subjected to centrifugation, sonication, or other physical or chemical processes (multiple washing steps, etc.) to assist in preparing the particles for detection. The microparticles may also be magnetic to facilitate collection and washing and other assay processing steps. In another example, the microparticles can be placed onto a glass slide or other specially prepared substrate for detection, or layered or otherwise applied across FFPE samples, etc. In yet other examples, the particles are present in a flow stream during detection, or present in a suspended solution.

Term conjugation is used to refer to the process by which substantially each microparticle has one or more probe molecules attached to its surface. Methods of conjugation are well known in the art, for example in Bioconjugate Techniques, First Edition, Greg T. Hermanson, Academic Press, 1996: Part I (Review of the major chemical groups that can be used in modification or crosslinking reactions), Part II (A detailed overview of the major modification and conjugation chemicals in common use today), and Part III (Discussion on how to prepare unique conjugates and labeled molecules for use in applications).

The molecular probes attached to the surface of the particles typically have known attributes or properties. In an example, the molecular probes can be derived from biological specimens or samples and used in the screening, including but not limited to genetic sequencing, of large populations where typically, the derivatives from one member of the population is applied to a single code, typically a multiplicity of particles of a single code. Preferably, microparticles having the same code have attached substantially the same probe molecules; whereas microparticles having different codes likewise have different probe molecules.

In the biological assays, fluorescent tags can be employed when an optical imaging method based on the presence of fluorescence can be used. Radioactive labels can be used when the microparticles are utilized to expose or develop relevant photographic films. Alternatively, enzymatic tags can be used when the detection involves detection of the product of the enzyme tag that is released when the sample molecules bind to or react with the probe molecules on the microparticles. Other tagging methods are also possible, as set forth in “Quantitative monitoring of gene expression patterns with a complementary DNA microarray” by Schena et al., Science, 1995, 270-467, the subject matter of which is incorporated herein by reference in its entirety.

Samples without labels can also be reacted with the microparticles. For example, molecular beacon probes can be applied to the microparticle. Molecular beacon probes typically contain a hairpin structure that, upon binding the non-labeled, or in some examples labeled, sample molecules unfold, thus producing a signal indicative of the binding events. (See, for instance, Marras et al., “Real-time assays with molecular beacons and other fluorescent nucleic acid hybridization probes,” Clinica Chimica Acta, 363:48-60, 2006, incorporated herein by reference in its entirety for all purposes). Such molecular beacon probes, as well as other probes, may be used in assays involving FRET (Fluorescence Resonant Energy Transfer), where for example fluorophores or quenchers are placed on or in the surface of the microparticles or the analytes being detected. Further, enzyme substrate FRET signals may be designed which remain quenched until acted upon by an enzyme. The enzymatic action on the substrate causes a conformational change or chemical change of the substrate which uncovers the FRET pair and produces signal.

Another preferred example of the invention is a kit comprising 200 or more encoded microparticles, more preferably 500 or more, 1000 or more, or even 10,000 or more different codes within the kit (due to the large codespace enabled by the invention, even larger numbers of codes). Due to statistical sample requirements of convenient liquid pipetting and a desired redundancy of particular codes within the kit, more than 10 particles of the same code are typically provided (20 or more, or even 30 or more microparticles of the same code) within the kit, as in some example applications the redundancy improves the overall assay performance.

Universal adapter schemes may be used to provide a set of non-interacting synthetic sequences that are complementary to sequences provided on the probes. Genotyping can be performed using common probes and allele specific reporters or allele specific probes and common reporters. Amplification assays such as those involving PCR, padlock probes, or Molecular Inversion Probe (MIP) technology can be performed using the particles of the current invention. In an alternative example of the invention, biomolecules that are present on the surface of the particles can be pre-synthesized and then attached to the particle surface. Alternatively, biomolecules can be in situ synthesized on the particles.

Protein based assays are also applicable. These include but are not limited to sandwich immunoassays, antibody-protein binding assays, receptor-ligand binding assays, or protein-protein interaction assays, and detection of transcription factor binding sites, as explained in more detail, below. The sets of encoded microparticles of the present invention can be used in solution based assays to investigate protein-protein interactions. A single type of protein can be applied to microparticles of a single code. Upon mixing of the particle-protein conjugates and reaction in a particular biochemical environment, proteins that interact and bind to one another are determined by the presence of adjacent particles during detection. Protein conjugation can be performed either directly by reacting free amines on the protein to available carboxyl groups on the particle, or indirectly through hybridization of complimentary oligonucleotide linkers or R-DNA or L-DNA. Additionally the direct protein conjugations may include one or more PEG-linkers to provide distance between the protein and the encoded microparticle surface. Nucleic acid conjugations may also utilize PEG-linkers. In all cases PEG polymers and/or polyvinyl-derivatives may also be used to passivate un-reacted particle surfaces and assist in “blocking” non-specific interactions.

The square cross section of the encoded microparticle structures of the present invention provides an improvement over the prior art by providing an increased area of contact in the shape of a flat, rectangular surface. Prior art particles that are spherical or cylindrical in shape limit the contact areas to single points or lines, respectively. Any interacting molecules may be used with this assay architecture. For instance, the flat surface of the present encoded microparticles are particularly well suited as a platform for efficient capture of whole cells, which may then be simply isolated if the encoded microparticles have magnetic properties.

The encoded microparticles may be used in conjunction with a 2D planar array of molecules. Interaction between molecules on the surface of the particles and those contained in spots on the 2D planar array are determined by the binding of the particles to the spots. The presence of the particles in the predetermined spot locations, preferably after washing steps, indicates a binding interaction between the molecules on the particles and the molecules on the 2D planar array. The assay result can be determined by identifying 1) the particle code, and 2) the spot location. The square cross section of the microparticles of the present invention provides for increased binding contact area compared with the prior art.

The microparticles of the invention may have other applications. For example, by placing protein-detection molecules, e.g., ligands, dyes which change color, fluoresce, or cause electronic signal upon contact with specific protein molecules, onto the microparticles, bioassay analyses can be performed, i.e., evaluation of the protein and/or gene expression levels in a biological sample or interactions thereof. As another example, by placing (cellular) receptors, nucleic acids/probes, oligonucleotides, adhesion molecules, messenger RNA (specific to which gene is “turned on” in a given disease state), cDNA (complementary to mRNA coded-for by each gene that is “turned on”), oligosaccharides and other relevant carbohydrate molecules, or cells (indicating which cellular pathway is “turned on”, etc.) onto the microparticles, the microparticles can be used to screen for proteins or other chemical compounds that act against a disease (i.e., therapeutic target); as indicated by (the relevant component from biological sample) adhesion or hybridization to specific spot (location) on the microarray where a specific (target molecule) was earlier placed/attached. In fact, the microparticles of the invention can be applied to many other biochemical or biomolecular fields, such as those set forth in the appendix attached herewith, the subject matter of each is incorporated herein by reference.

It will be appreciated by those of skill in the art that a new and useful microparticle and a method of making the same have been described herein. The large sets of encoded microparticles produced by this invention are a fundamental technology that may have very broad applications, especially in fields such as biotechnology, pharmacology, microbiology and specifically genomics. Utilization of the presently described encoded microparticles in biological assays has the potential to dramatically reduce the cost of highly multiplexed bioassays. Moreover, the presently described encoded microparticles allow researchers to easily design custom content solution arrays at a low cost. The investigator/user can also easily add new particle types to the pooled set, for instance including new found genes of interest with the microparticles of the invention.

In view of the many possible embodiments to which the principles of this invention may be applied, however, it should be recognized that the embodiments described herein with respect to the drawing figures are meant to be illustrative only and should not be taken as limiting the scope of invention. Those of skill in the art will recognize that the illustrated embodiments can be modified in arrangement and detail without departing from the spirit of the invention.

### Encoded Microparticles to Detect Analytes

The presently disclosed encoded microparticles may be utilized, for instance, in detection of protein-protein interactions and/or isolation or purification of proteins or proteinaceous components through protein-protein interactions. The microparticles are composed of a substance which allows attachment of various biochemical components which are capable of binding analytes.

For instance, although it is known that oligonucleotide probes may be bound to such encoded microparticles. For each code in a group of encoded microparticles, a specific oligonucleotide probe comprising a specific gene sequence may be bound. A labeled genetic sample may then be processed, for instance by restriction digest and amplification and labeling, and then exposed to the encoded microparticles such that the labeled genetic sample fragments may then bind or hybridize to the encoded micoparticles. Once bound, the encoded micoparticles are thereby labeled and may be detected. By reading the code of the labeled encoded micoparticles, one can determine which genetic sequence were, or were not, present in the sample. This is highly advantageous for detecting various mutations and other genetic abnormalities which may be indicative or predictive of disease states.

On the other hand, the encoded micoparticles may also be bound to other probes, such as a proteinaceous component which is specific for a type of ligand to be analyzed within the sample, or purified from the sample, or quantitated in some manner. Such types of ligand-receptor or affinity interactions may include, but are not limited to, lectins and other components which bind sugar molecules, antibodies and antigens, receptors and ligands, aptamers, agglutinins, enzymes and substrates, and the like. In this manner, the encoded micoparticles acts as a surface upon which the desired analyte, or probe, may be precipitated, or “fished” out of solution.

The probe may itself be labeled, as in the oligonucleotide example, above, or the analyte may be labeled. If the analyte is unlabeled, a second binding partner may be introduced which has affinity for the analyte and is labeled.

The encoded micoparticles bound to analyte may be further detected or labeled either in solution without any further purification, or they may be isolated and washed and then detected. It is likely that in some assays, optimization may require first purifying the encoded microparticles before detection. Purification of the encoded microparticles may be easily accomplished if the encoded microparticles are magnetic. Addition of a magnetic substance or layer in the manufacture process may be achieved, as discussed in the cited references above, by use of standard methods and magnetic materials.

By purification of the encoded microparticles at any step in the assay, the analyte may also be purified and released in pure form. That is, once analyte is bound to the encoded microparticles by interaction with the probe molecule, the encoded microparticles may then be purified with the analyte attached. The analyte may then be released from the encoded microparticles through exposure to a competitor of the analyte, or dialysis, or a change in the buffer conditions, such as an increase or decrease in salt concentration, or change in pH, thereby releasing the analyte. Once analyte is released, the encoded microparticles may be removed from solution by use of magnets if they are magnetic, or may be spun down and precipitated by centrifugation, or filtered out of solution by passing the solution through a filtering apparatus such as filter paper or a size exclusion chromatography column, or other known chromatographic techniques.

One means of labeling the analyte, once bound to the encoded microparticles, is by way of a secondary antibody. Much like an ELISA or sandwich immunoassay, the encoded microparticles may be bound to various primary antibodies which are specific for a first antigenic determinant on the target. Once antigen is bound, a secondary antibody, specific for a second, different antigenic determinant on the target, may be bound to the encoded microparticle-target complex, thereby creating an encoded microparticle-antibody-target-antibody complex. The secondary antibody could be labeled, thereby labeling the encoded microparticles to which unlabeled target is bound. Alternatively, there may be multiple different targets, some labeled and some unlabeled, which then may be bound in the same manner to the encoded microparticles and then alternately labeled and/or bound by a secondary antibody to detect the bound analyte. Similar assays exist in the market, but lack the additional features and advantages provided by the encoded microparticles of the present biochemical assays. (See, for instance, the Barcoded Magnetic Bead (BMB) products at Applied BioCode, Inc., Santa Fe Springs, Calif., website with proposed Immunoassay, see also U.S. Pat. Nos. 6,949,377, 7,122,153, 7,858,307, 7,871,770 and 7,339,153 and U.S. Patent Application Publication Nos. US 20020127740, US 20070170353, US 20090061507, US 20110007955, and US 20110152127, all of which are incorporated herein by reference in their entirety for all purposes).

The above embodiment may be employed also using lectins or other binding partners, so long as the binding partners are capable of forming a ternary complex wherein one member of the complex is also labeled, as above. In another embodiment, the encoded microparticles themselves may be placed into an empty chromatography tube and thereby create a chromatography column out of the encoded microparticles which have bound thereto the analyte of interest. Analyte may be released any number of ways, consistent with standard affinity chromatography techniques. The microparticles may also have attached thereto various different oligonucleotides for the purpose of binding to DNA and/or RNA binding proteins. This assay may also be used to purify such DNA and/or RNA binding proteins for use in further assays.

Other applications of this technology include real-time monitoring of analytes during an experiment or product preparation. In one embodiment, the encoded microparticles are bound to single chain FRET molecules, or biosensors. A biosensor has attached to it two FRET molecules. When the two molecules are within close proximity to each other, within about 60 \acute{Å}, the fluoresce. The concept of FRET works on the basis of two fluorophores paired together; one fluorophore possesses an emission wavelength which overlaps with the excitation wavelength of the second fluorophore. Therefore, when the first fluorophore is excited, it simultaneously excites the second fluorophore if the second fluorophore is close enough to be excited by the emission fluor of the first fluorophore. Scientists have been able to modify a single molecule, such as a protein chain, to covalently attach both molecules of a FRET pair. Furthermore, the FRET signal only occurs when the single molecule is placed into a specific conformation, i.e. the molecule undergoes a conformational change upon binding of its ligand which then brings the paired FRET molecules into close proximity and causes the FRET signal to be emitted. (See, for instance, Cell Migration Consortium, Cell Migration Gateway, “Biosensors—Approaches” EISSN 1747-7883, incorporated by reference in its entirety for all purposes).

This approach allows detection and quantification of analytes that are otherwise unlabeled. The binding of the analyte to the FRET single chain molecule causes a conformational change which then elicits a signal which may be detected in real time. Other environmentally-sensitive fluorophores have also been reported, such as PRODAN (6-propionyl-2-(dimethylamino)naphthalene) and reported derivatives thereof, 4-aminophthalimide and 4-N,N-dimethylamino phthalimide (DMAP), various benzofuran derivatives such as Fmoc-Dap-OH(NDB) and RTI-233. (See, Wouters et al., 2001, “Imaging biochemistry inside cells,” Trends Cell Biol., (5):203-11, Zhang et al., 2002, “Creating new fluorescent probes for cell biology,” Nat Rev Mol Cell Biol., (12):906-18, Weber et al., 1979, “Synthesis and spectral properties of a hydrophobic fluorescent probe: 6-propionyl-2-(dimethylamino)naphthalene,” Biochemistry, 18(14):3075-8, Vazquez et al., 2003, “Fluorescent caged phosphoserine peptides as probes to investigate phosphorylation-dependent protein associations,” J Am Chem. Soc., 125(34): 10150-1, Cohen et al., 2002, “Probing protein electrostatics with a synthetic fluorescent amino acid,” Science, 296(5573):1700-3, Nitz et al., 2002, “Enantioselective synthesis and application of the highly fluorescent and environment-sensitive amino acid 6-(2-dimethylaminonaphthoyl)alanine (DANA),” Chem Commun (Camb)., (17):1912-3, Valeur, B., “Molecular Fluorescence, Principles and Applications,” Wiley-VCH, Weinheim, Federal Republic of Germany, 2002, Saroja et al., 1999, J. Phys. Chem. B, 103, 2906, Rasmussen et al., 2001, “Biophysical characterization of the cocaine binding pocket in the serotonin transporter using a fluorescent cocaine analogue as a molecular reporter,” J. Biol. Chem., 276(7):4717-23, all of which are hereby incorporated by reference in their entirety for all purposes).

These technologies, combined with the encoded microparticles, may be employed in conducting real time multiplex assays. For instance, during fermentation of a recombinant microorganism, one may obtain samples of the fermentation liquid as fermentation progresses. Samples may be taken in aliquots at certain times and then optionally lysed and bound to the encoded microparticles. If the encoded microparticles have bound them already a single chain FRET molecule which fluoresces upon binding or other type of biological event, such as activation, then the fermentation sample may be analyzed for that event, i.e. the analyte which binds to the single chain FRET molecule. If the analyte or analytes exist in the fermentation broth, i.e. the microorganism secretes the analyte(s), then no lysis is required and the broth may be immediately incubated with the encoded microparticles and the microparticles then analyzed/detected with reflected light to read the code and fluorescence to detect the FRET signal.

There recently have been reported newer FRET techniques that are equally applicable for encoded microparticle assays. The new techniques are referred to as fluorescence-lifetime imaging microscopy (FLIM). This family of techniques can be sub-divided into two groups, frequency domain FLIM or time domain FLIM. FLIM is an imaging technique for producing an image based on the differences in the exponential decay rate of the fluorescence from a fluorescent sample. For instance, in frequency domain FLIM a laser is used to excite the fluorescent label, but in a pulse mode. That is, the laser is operated in pulses of light, and the decay pattern of the fluorescent label is measured. The decay pattern of the fluorescent label changes, or shifts, depending on the microenvironment of the fluorescent label. An LED light source may be used, operated in pulse mode, i.e. using a modulated LED source (commercially available from companies such as Thorlabs, Newton, N.J., US). In FLIM experiments, the camera is operated using a gated intensifier which matches the pulse frequency of the light source enabling sampling of the decay pattern between the light pulses. Such cameras may be capable of taking 7-9 pictures between a single pulse, depending on the speed and sophistication of the camera. FLIM may be conducted in combination with FRET techniques. Thus, the FLIM measurement will tell the investigator whether the fluorescent molecule is fluorescing or undergoing FRET interaction, which also informs the investigator about the microenvironment of the label, which in turn teaches the investigator about the interaction of the label. Most importantly, the FLIM technique may be used in assays where only one of the interacting species is labeled, i.e. either the target or probe is labeled, etc. (See, for instance, Wallrabe et al., “Imaging protein molecules using FRET and FLIM microscopy,” Curr. Opin. Biotechnol., 16(1):19-27, 2005; Laptenok et al., “Fluorescence Lifetime Imaging Microscopy (FLIM) Data Analysis with TIMP,” J. Stat. Software, 18(8):1-20, 2007; Swift et al., “Basic Principles of FRAP, FLIM and FRET,” Proc. Royal. Mic. Soc., 39:3-10, 2004; and Lambert Instruments Perfection in Image Detection publication, “Fluorescence Lifetime Imaging Microscopy,” available from Lambert Instruments B.V., The Netherlands; all of which are incorporated herein by reference in their entirety for all purposes). In these experiments, the label may be attached to the microparticle, the antibody or probe or molecule being used to detect the analyte, or the analyte/binding partner of the assay.

In other embodiments, the solution containing the analyte could be doped with encoded microparticles having bound thereto the single chain FRET molecule, or other detector. I continuous stream of analyte may be withdrawn from the biological assay and sent through a flow cell whereby the code is read on the encoded microparticle and simultaneously, or nearly so, the fluorescence signature of the microparticle also determined. Thus, in real time one can perform a multiplex assay of several analytes with no labels needed other than the single chain FRET molecule, or other environmentally-sensitive fluorescent trigger bound to the encoded microparticle.

The entire assay may further be automated. For instance, if a large batch of chemical were being synthesized in commercial quantity, the encoded microparticles may again be doped into the solution and be in solution with the reactants as the reaction progresses. A small stream of solution from the reaction vessel (which could be as large as 100 to 1000 liters or more, or as small as 0.25 to 1 liter or less) may be continuously withdrawn through a tube or other means and sent through a flow cell whereby, as above, the encoded microparticles may be “decoded” by reading their codes as they fly by the detector, and simultaneously (or nearly so) the fluorescence signature be read. In this manner, one may follow the course of the reaction as one or more of the substrates are depleted and/or one or more products (or side-products or by-products, as the case may be) are formed. All of the substrates and/or products may be detected simultaneously in such an assay, optionally in real time.

In the multiplex assays described herein, each code of the encoded microparticles would correspond to a different FRET (or other) sensor molecule and therefore a different analyte. Furthermore, by adjusting the flow rate through the flow cell and knowing the volume thereof, one can easily determine concentration of analytes and chart progression of the reaction or fermentation or other biological event as it occurs.

Examples of creating such biosensors, i.e. single-chain FRET pairs, are available in the literature. For instance, see De Lorimier et al., “Construction of a fluorescent biosensor family,” Protein. Sci., 11(11):2655-2675, 2002 and Sekella et al., “A biosensor for theophylline based on fluorescence detection of ligand-induced hammerhead ribozyme cleavage,” RNA, 8:1242-1252, 2002, and references cites therein, incorporated herein by reference in its entirety for all purposes.

In another embodiment of the above, the encoded microparticles may be injected into a subject and used to measure the progression of disease, the half life of pharmaceuticals also injected to the subject, the effect of a treatment on a patient by measuring disease indicators, and the like.

In another embodiment of this type of assay, the analyte may be labeled with one FRET molecule and the specific binding partner of the analyte bound to the encoded microparticle labeled with the second FRET pair molecule. Thus, rather than rely on a conformational change, environmental quality or activation/deactivation process as in single-chain FRET molecules, separately tagging the encoded microparticle molecules with one of the FRET pairs and the analyte with other offers other advantages. For instance, a purified analyte of interest could be labeled and studied in a multiplex manner. One example of such an assay would be determination of an epitope for an antibody. Each differently encoded microparticle in the assay may be bound to a possible epitope and the encoded microparticle also bound with one of a FRET label pair. Antibodies could then also be labeled with the other label of a FRET label pair and the encoded microparticles and antibodies incubated together. Binding of the antibody to the encoded microparticle with the correct epitope would yield a detectable FRET signal. The encoded microparticle could then be decoded and the sequence of the epitope determined. In this assay, epitope mapping may be conducted in a rapid and efficient multiplexing format for many antibodies and many epitopes, optionally in a high-throughput manner.

The epitope to be mapped on the encoded microparticles may be any kind-of epitope known in the art. For instance, most common epitopes are protein-based, comprised of amino acids. However, antibodies are also known to bind to nucleic acids, oligonucleotides, nucleic acid analogs, cofactors, vitamins, circulating cells, adherent cells, neurotransmitters and other cell signaling molecules, lipids, biotin, carbohydrates, drugs, and other molecules. All of these molecules may be bound to the encoded microparticles in various orientations or in small pieces, i.e. the molecule may be broken apart into sub-components and those sub-components then each bound to a different encoded microparticle. Detection and analysis of the encoded microparticles bound to antibody may then be conducted as described above either statically in a container as the particles settle or by flow analysis, for example.

The encoded microparticles may also be used in a “two-hybrid” type of assay. (See, Fields et al., “The two-hybrid system: an assay for protein-protein interactions,” Trends in Genetics, 10(8):286-292, 1994, incorporated herein by reference). Use of the encoded microparticles in such a manner would be simpler and easier than the traditional two-hybrid system because the entire experiment may be conducted in vitro without need for transfection or use of microorganisms. For instance, a library of proteins may be expressed in vitro, then purified and labeled. This library may then be contacted with the encoded microparticles having a specific protein of interest for which binding partners are to be identified. The encoded microparticles may either be contacted with the in vitro translated protein library in a container and mixed, or the encoded microparticles may be bound to a surface in a particular order such that those particles which have labeled protein bound thereto may be isolated and analyzed, and the protein bound also be analyzed and possibly sequenced to determine identity. This assay may be made simpler if only a couple potential binding partners are desired to be analyzed. In this manner, the small number of potential binding partners may each be differently labeled and thereby detected in the same manner as above.

As already briefly discussed above, the encoded microparticles may be used to detect the presence of microorganisms and/or viruses in a sample. By binding different antibodies each with a different specificity to each encoded microparticle having a specific code, the encoded microparticles may then be incubated with a sample which has been labeled. Subsequent isolation and washing steps will reveal specific encoded microparticles with antigen bound thereto. Once decoded, i.e. the chart of different codes is consulted to determine which antibody having which specificity is associated with the microparticle code, the presence of the antigen in the sample may be conclusively deducted. In this multiplex assay, samples may be tested for a multitude of infectious disease causing agents in a single assay using very little reagents and material. Purification and washing may be optional steps with the present assay with conditions optimized for reading of the encoded microparticles in the non-purified samples.

Real-Time Monitoring of Biological Events with Encoded Microparticles

The presently disclosed encoded microparticles may be utilized, for instance, in detection of cell-cell interactions, cell growth, cellular secretions and inter-cellular signaling using the encoded microparticles as detectors of specific analytes which may be transiently present during these events. In one embodiment, the encoded microparticles may be embedded into a matrigel material and a cell sample placed on the gel and grown as in a tissue culture. The encoded microparticles may have attached thereto various biosensors, as described above, designed to detect the presence of intercellular signaling molecules, secreted molecules, and other cell development and other biochemicals.

The gel may be any matrigel or gel or other matrix which is commercially available and capably of sustaining cell growth. Examples include BD MATRIGEL™ Matrix available from Becton Dickinson, BD Biosciences, US. For instance, see also Lang et al., “Differentiation of prostate epithelial cell cultures by matrigel/stromal cell glandular reconstruction,” In Vitro Cell Dev. Biol. Anim., 42(8-9):273-280, 2006, and Hughes et al., “Matrigel: a complex protein mixture required for optimal growth of cell culture,” Proteomics, 10(9):1886-1890, 2010, incorporated herein by reference.

By strategically positioning the encoded microparticles at the bottom of the tissue culture surface, the encoded microparticles may be detected through the tissue culture surface and label signal measured in real time. In other embodiments, small samples may be taken over time, by extracting a piece of the matrigel and solubilizing the embedded encoded microparticles and then detecting the encoded microparticles. Alternatively, a standard tissue culture solution may be used in these assays with the encoded microparticles being on the bottom of the tissue culture container and the agar or other gel/fluid being on top of the particles.

Alternatively the cells may be cultured on removable inserts containing permeable membranes containing various coatings and growth factors, where the particles rest at the bottom of the well below the inserts. The inserts may be different sizes to match the well diameter of various micro-titer plates ranging from 12-well to 96-well plates. The plates may be glass or have optically transmissive bottoms exhibiting low auto-fluorescence and thicknesses near a standard #1.5 coverslip (0.17 mm). These membranes also have the potential for compatability for non-adherent suspension cells. The particles may be imaged inside the plate, from underneath the plate, using an inverted microscope or scanner based on an inverted microscope design that utilizes fluorescence in the lightpath.

Such methods may be utilized to measure real time, or snapshots in time, events which occur during cell growth, propagation, differentiation and other important biological events. The progress of infection may be followed or even the differentiation of tissues and organs using this technique.

Labeling and Sorting of Specific Cell Types with Encoded Microparticles

The presently disclosed encoded microparticles may be utilized, for instance, in detection and optionally labeling of specific types of cells in a heterogeneous population of cells. Upon labeling of multiple specific cell types with the encoded microparticles, they may be visualized and optionally, if circulating, sorted using commercially available cell sorting instruments and techniques.

This type of multiplex assay is easy to manipulate for the specific purpose/assay desired. In general the encoded microparticle may be bound to the antibody of choice, i.e. an antibody specific for any known cell marker. Common cell markers include receptors, such as the CD family of markers. Generally cell markers are designated a “CD” number used to identify the marker/receptor/protein in the literature. For instance, CD50 is also commonly known as ICAM-3, the third counter receptor for LFA-1 (which is CD54). The abbreviation “CD” stands for “cluster of differentiation” and is meant to indicate the immunophenotype of a cell. CD molecules may be either receptors or ligands, depending on the CD number and some CD molecule play critical biological roles in events such as cell signaling and adhesion. (See, Zola et al., “CD Molecules 2006—Human Cell Differentiation Molecules,” J. Immunol. Meth., 318(1-2):1-5, 2006, incorporated herein by reference). Immunophenotyping allows investigators to identify the type of cell present by way of which CD molecules are present at the surface of the cells. These CD molecules are typically detected by binding antibodies specific for the CD molecule of interest to the cells and then sorting the cells using FACS analysis (flow cytometry), or otherwise visualizing or detecting the bound antibodies.

The encoded microparticle may similarly be utilized to identify a multitude of different cell types in a single assay using a single type of label. In this assay, each differently encoded microparticle has attached to it a labeled antibody possessing a specificity for a cell-determinant antigen (such as a CD molecule). Many different encoded microparticles, each having a different code and each having bound thereto a labeled antibody with a unique specificity for a particular antigen, may be incubated together with a mixed cell population. The encoded microparticles will then bind to the specific cells of interest and they may then be “decoded” as above and the cell types present in the sample determined using a flow cytometry instrument (such as an Amnis ImagestreamX high resolution microscopy in flow system, for example, Amnis Corp., Seattle, Wash., US) or by simply looking at the sample under a microscope or other scanning instrument when the cells are in a container to detect both the code and the presence/absence of a label. This may be accomplished using the usual techniques, such as using a brightfield microscope in either an upright or inverted design, in brightfield or transmission mode, or on phase contrast mode, or in differential interference contrast mode, or in polarization contrast mode. The encoded microparticles may have attached to their surfaces additional amine and carboxyl functional groups which facilitate cross-linking using formaldehyde or para-formaldehyde solutions with or without 1-ethyl-3-[3-dimethylaminopropyl]carbodiimide hydrochloride (EDC). Cells may be further permabilized by methanol or saponin fixation and the QuantiGene® View DNA/RNA assay performed to correlate CD marker with SNP, CNV, or gene expression patterns.

The cells having the encoded microparticles bound thereto may be isolated any number of ways, especially if magnetic particles are used. If the encoded microparticles are magnetic, those cells having a microparticle bound to them will be isolated along with the encoded microparticles as they are pulled down by their magnetism. In this way, a specific population of cells may be isolated or enriched in a sample.

Furthermore, this assay may also be applied to fixed tissue samples, such as FFPE (formalin fixed paraffin embedded) samples. The encoded microparticles, having labeled antibodies bound to them as described above, may be washed over such a tissue sample, for instance on a slide, and those that specifically bind will not be washed away under the proper conditions. The microparticles that remain bound can then be scanned and visualized and their codes detected. In this way, tissue sections may be efficiently labeled based on the types of cells present in different areas of the tissue. Again, these assays may be performed in a multiplex manner in that multiple different coded microparticles will be used, each different code having bound to it a different labeled antibody possessing a single specificity for a certain molecule which is determinative of the cell type, tissue type or other characteristic of a cell.

This assay may make use of any of the labels described above, such as the biosensors and other environmentally sensitive labels described herein and elsewhere in the literature. Furthermore, the molecule bound to the encoded microparticle, i.e. the “probe”, need not be an antibody. It is known that many cells possess a surface coated with antibodies and/or receptors whose binding partner may be attached to the surface of the encoded microparticles. For instance, specific cells may possess specific MHC molecules with binding specificity for particular antigens. Thus, cells expressing MHC molecules may be identified based on antigenic specificity by attaching a library of labeled antigens to the encoded microparticles (either the antigen may be labeled or the encoded microparticle itself otherwise labeled). Likewise, various carbohydrates may be attached to the microparticles and the microparticles then used to bind to and identify those cells possessing receptors which specifically recognize the various combinations of carbohydrates attached to the microparticles. These are merely a few exemplary embodiments of the types of biological assays that may be performed utilizing the encoded microparticles and are not in any manner meant to limit the presently disclosed invention.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of detecting an analyte, which comprises:
providing a set of encoded microparticles which comprises two or more subsets of encoded microparticles, each of the two or more subsets of encoded microparticle comprising a spatial code unique to each subset;
attaching to each subset of the two or more subsets of encoded microparticles a unique probe possessing a specificity for a unique analyte, wherein either the unique probe or the unique analyte is labeled;
incubating the encoded microparticles with a sample suspected of comprising an analyte;
detecting the label;
determining the code associated with the detected label; and
identifying the presence of the analyte based on the unique probe corresponding to the determined code of the encoded microparticle.

2. A method of identifying a cell, which comprises:
providing a set of encoded microparticles which comprises two or more subsets of encoded microparticles, each of the two or more subsets of encoded microparticle comprising a spatial code unique to each subset;
attaching to each subset of the two or more subsets of encoded microparticles a unique probe possessing a specificity for a unique analyte, wherein either the unique probe or the unique analyte is labeled, and wherein the unique analyte is on a cell surface; incubating the encoded microparticles with a sample comprising or suspected of comprising the cell; and
detecting the presence of the label, thereby identifying the cell.
